(19) United States
(12) Patent Application Publication
(10) Pub. No.: US 2009/0221922 A1
(43) Pub. Date: Sep. 3, 2009

(54) METHODS AND DEVICES FOR IN SITU TISSUE NAVIGATION

(75) Inventors: Ryszard Lec, Philadelphia, PA (US); Mark R. Goodwin, Gilbertsville, PA (US); David Greg Anderson, Moorestown, NJ (US); Daniel Schwartz, Teaneck, NJ (US); Denis Drummond, Gladwyne, PA (US)

Correspondence Address: Pabst Patent Group LLP, 1545 PEACHTREE STREET NE, SUITE 320, ATLANTA, GA, 30309, US

(73) Assignees: Biospinex, LLC; Drexel University

(21) Appl. No.: 12/396,667

(22) Filed: Mar. 3, 2009

Related U.S. Application Data
(63) Continuation of application No. PCT/US09/35695, filed on Mar. 2, 2009.
(60) Provisional application No. 61/033,225, filed on Mar. 3, 2008.

(51) Int. Cl. A61B 6/00

(52) U.S. Cl. 600/478

(57) ABSTRACT

The “Smart Tool” includes a “Smart Tool Probe” and two processing modules. The Smart Tool Probe is a hand held, wired or wireless, device that a surgeon utilizes for interrogating and identifying a tissue site, such as the entrance to a pedicle. The processing units, an Electro-Optical Control (EOC) Module and a CDS Module, provide control and display capabilities enabling real-time tissue site (such as vertebra bone) interrogation. The Smart Tool Probe utilizes a system of optical fibers that carry the interrogating optical signal sent by the light source(s) and the reflected optical signal back to the optical receivers. The light source(s) and light receivers are located in the EOC Module. The data received from the EOC Module are processed and converted into an image which is displayed on the screen in real-time. The software installed on the machine allows the surgeon to adjust/enhance the image properties to suit the selected requirements. This mode of operation provides interactive image sharpening (to adjust image sharpness), threshold control (to adjust image contrast), segmentation (to delineate the density map in the image), and image calculus (to pin-point the center of a particular region in the image).

### CROSS-REFERENCE TO RELATED APPLICATIONS

This application claims priority to U.S. Ser. No. 61/033,225 entitled “Methods and Devices for In Situ Tissue Navigation” by Ryszard Lee, Mark R. Goodwin, David Greg Anderson, Daniel Schwartz, and Denis Drummond, filed on Mar. 3, 2008, and is a continuation of PCT/JS09/35695, filed Mar. 2, 2009, which also claims priority to U.S. Ser. No. 61/033,225.

### FIELD OF THE INVENTION

The current invention relates to the field of medical instruments and more specifically to devices and methods of use thereof to interrogate bone for the purpose of medical diagnosis or as a surgical tool in the placement of spinal implants during surgery.

### BACKGROUND OF THE INVENTION

Many problems in modern medicine require diagnostic information of the quality, makeup and substance of the bone of the skeleton. For example, osteoporosis is a bone weakening disease, affecting millions of people around the world. Early diagnosis and treatment of this disease are paramount to an optimal clinical outcome. In another example, tumors commonly develop or metastasize to the skeleton. The precise localization of tumor(s) within the bone is a crucial part of staging and treatment of the disease.

In another example, the detection of an un-united fusion graft, usually marked by a thin gap within the fusion mass that is filled with fibrous scar tissue, may be difficult to locate precisely. This situation can lead to pain with movement similar to that experienced with a fracture of a long bone and may require surgical treatment. However, accurate treatment is based on the precise location of the region of abnormal fibrous tissue within the bone, which may not be obvious on visual observation. In this case, precise interrogation would be desirable.

Another area requiring accurate interrogation of skeletal bone occurs during surgery for bony conditions. Many skeletal conditions require the placement of implants, which must be correctly positioned to optimally address the surgical condition. An example of the challenges encountered during skeletal surgery is demonstrated in reconstructive surgery of the spine, where metallic implants are placed to assist in the healing of a bony fusion or to correct and stabilize the deformed or disrupted vertebral column. During complex spinal surgery, e.g. fusion and non-fusion, metallic implants are often placed in the spinal pedicle, a narrow column of bone that connects the dorsal (back) portion of the spine to the vertebral body (front). Although the pedicle is an excellent anchor point from a biomechanical perspective, delicate tissues including the spinal cord, nerve roots and major blood vessels (aorta and vena cava) surround it. All of these structures can be injured during implant placement, leading to catastrophic consequences for the patient and with associated medical-legal implications.

Current surgical technique for the placement of implants into the spinal pedicle involves the placement of a pilot hole over the top of the column of the spinal pedicle, followed by cannulation (drilling of a bony passage) with a blunt instrument prior to placing the pedicle screw implant. To perform this procedure correctly, a surgeon must determine the point to open on the surface of the bone, directly over the bony column of the spinal pedicle and then must pass the instruments used to open the hole or passage through the spinal pedicle along a correct trajectory from back to front through the softer cancellous bony core of the pedicle column. If the trajectory or starting point is not accurate, there is a high risk that the shell of harder cortical bone may be breached, exposing the adjacent spinal cord, nerves or blood vessels to potential damage. Once a correct pilot hole through the pedicle has been achieved, the pilot hole can be expanded with a screw tap and a correctly sized pedicle screw can be safely placed in the hole.

Numerous examples of pedicle implants and systems thereof are known. See, for example, U.S. Pat. Nos. 6,488,681, 6,423,065, 6,312,431, 6,858,030, 7,163,539, 7,311,713, and patents cited therein. Conventional pedicle cannulation is essentially a “blind procedure”, meaning the surgeon cannot visualize the starting point or passage of the instrument during the process. Instead, the surgeon must rely on a combination of an understanding of the normal spinal anatomy plus tactile feedback to achieve correct placement of the implant.

To assist spinal surgeons in placing spinal implants, conventional radiographic imaging technologies have been used, the most basic of which is a fluoroscopic C-arm. This portable x-ray unit can be used to visualize the two-dimensional anatomy of the spine in relation to the instrument that the surgeon is using to make the pedicle passage. However, the use of radiographic imaging provides only a two-dimensional picture of the complex three-dimensional anatomy of the spine and exposes the surgical team and patient to potentially large amounts of ionizing radiation. In addition, the equipment is bulky, cumbersome to use, and requires a dedicated technician to operate.

More sophisticated imaging techniques have been developed to assist with spinal implant placement, including computer-assisted image guided surgery. These systems use pre-acquired images, not real-time images, from either fluoroscopy or computed axial tomography (CAT) scanning that, in combination with software, can be correlated to the patient's anatomy during surgery. To accomplish this, a reference array must be securely attached to the patient's anatomy and to any instruments used for the pilot hole and pedicle cannulation phase of the surgery. A computer then tracks the position of the instruments relative to the pre-acquired images and gives the surgeon a “virtual” picture of where the instruments are in relation to the spinal anatomy.

Although this approach seems to be appealing, the use of computer-assisted surgery during spinal surgery has been fraught with difficulties that have limited its use. For example, the set up and use of the equipment is cumbersome and highly technical. Additionally, the equipment is bulky and sensitive to being accidentally “bumped” during the procedure, dislodging the reference array attached to the spine and rendering the navigation unreliable and inaccurate. Further, the equipment is expensive and generally requires a dedicated technician for successful use. Most surgeons have found that the lack of “real time” data prevents them from routinely trusting the navigated images for the placement of complex implant constructs. Also, all of the developed navigation techniques are only focused on image guidance through the pedicle once the entrance to it has been identified. None are able to identify the entrance that is hidden deep within a cortical bone cover. Accurate identification of this stating point is arguably the key to successful and time efficient navigation. Finally, computer assisted systems have been found to increase operative times and the cost of surgery. Therefore, many institutions where complex spinal surgery is performed have abandoned the use of computer assisted systems for spinal procedures.

Although spinal surgeons have become increasingly good at understanding the complex anatomy of the spine, studies have documented that approximately 15-20% of pedicle screws are not correctly placed. Reasons for incorrect placement of pedicle screw implants include variations in spinal anatomy between individuals, altered spinal anatomy as a result of disease, trauma or deformity of the spine, poor or misleading radiographic images of the spine, small pedicles, obesity, bony overgrowths from the joint obscuring the starting point, and/or poor bone quality. These factors can make the identification of the pedicle starting points and trajectory difficult to identify even by experienced spinal surgeons.

Devices that can be used to image the area into which an implant is to be placed are described in U.S. Pat. Nos. 6,579,244 and 6,849,047 to Goodwin, describing a probe for imaging before, during or after placement of a screw, and U.S. Pat. No. 6,719,692 to Kleffner, describing a drill or cutting instrument that allows imaging during cutting, both preferably using ultrasound. There is only one other navigation system that uses a hand held instrument, PEDIGUARD®. See European Spine Journal, 12(1), 2003. This device uses bone impedence to provide data. It makes no attempt to find the starting point (ie. define the entry to the pedicle that is hidden by cortical bone). Thus PEDIGUARD® only navigates once the instrument is in the pedicle, and that is a key issue. Basically it is best in finding a breach that has already been made.

Clearly, a better method and tool is needed to interrogate skeletal bone to be used for diagnostic purposes as in osteoporosis, bone cancer, non-unions, (pseudarthrosis), infections and as an aid during complex bone surgery such as the placement of pedicle screws into the spine.

Therefore, there is a need for improved methods and tools for interrogation of skeletal bone.

It is an object of the invention to provide improved methods and devices for interrogation of skeletal bone.

It is a further object of the present invention to provide a simple, accurate and “user friendly” method to interrogate the substance of an intact skeletal bone.

It is a further object of the present invention to provide simple and accurate feedback to a surgeon on bone properties before, during and after the surgery.

It is a further object of invention to identify fibrocartilage within a bone fusion mass, following surgical arthrodesis (fusion procedure), indicting a failure of solid fusion or pseudarthrosis.

Another object of the present invention is to use light energy with the capacity to penetrate bone variably based on the substance of the underlying bone (mineral density, thickness, characteristics of the bone marrow, hardness, micro-architecture, tissue infiltration, and blood flow).

Another object of the invention is to provide a device with the capacity to emit light and to detect the reflected and scattered light energy that returns to the device and to transduce the data contained in the reflected and scattered light as a means to quality the underlying bone.

Another object of the present invention is to use light wave of variable intensity to penetrate bone at different depths, enabling three-dimensional image representation of the bone/vertebra structure.

Another object of the present invention is to use light wave of variable intensity to penetrate bone at different depths to implement bone image slicing capabilities at the depth of interest of the bone/vertebra structure.

A further object of the invention is to provide a device providing information regarding bone mineral density in cases of osteoporosis/osteopenia or osteopetrosis.

A further object of the invention is to provide a device providing information regarding bone structural features such as bone integrity, fracture, dislocations, etc.

Yet another object of the invention is to provide a device capable of differentiating the location, extent and qualities of a bone tumor, both primary and metastatic.

Yet another object of the invention is to provide a device capable of detecting fibrous tissue with the substance of bone in order to detect a non-union or pseuarthrosis.

Yet another object of the invention is to provide a device capable of imaging bone during surgical procedures where bone resection (cutting and removing) is required to assist in the accuracy of the bone cuts.

Yet another object of the invention is to provide a device capable of localizing an introduced therapeutic medical device (e.g. needle, wire, or catheter) in relation to the underlying bony substance.

Yet another object of the invention is to provide a device capable of detecting the proper or optimal position for a bone implant to be placed.

Yet another object of the invention is to provide a device capable of detecting the proper starting point and trajectory to be used for the cannulation and placement of a screw or pedicle implant into a spinal pedicle.

Another object of the invention is to provide a device capable of evaluating effectiveness or correctness of the surgery or a screw placement during and after a surgery.

Still another object of the invention is to provide a device capable of real-time comparison of the surgery-in-place with a library of images taken during past surgeries.

### SUMMARY OF THE INVENTION

A surgical probe device, and system for use thereof, containing a light emitting source, high-fidelity optical position sensor, signal conditioner and a telemetry method for data transmission to the medical practitioner or team, is used for the non-invasive interrogation of bone, providing real-time data on the bony substance. The device does not require a dedicated technician to operate it, provides high accuracy, no ionizing radiation exposure to the medical team or patient, and is inexpensive to manufacture. The device emits light onto or into a bony surface which is variably absorbed by the underlying bony substance. A portion of the light is reflected and scattered back to the device according to the intrinsic properties of the bone. The reflected and scattered light is detected and the data is processed to provide “real time” information of the bone adjacent to the tip of the instrument.

The “Smart Tool” includes a “Smart Tool Probe” and two processing modules. The Smart Tool Probe is a hand held, wired or wireless, device that a surgeon utilizes for interrogating and identifying a tissue site, such as the entrance to a pedicle. The processing units, an Electro-Optical Control (EOC) Module and a CDS Module, provide control and display capabilities enabling real-time tissue site (such as vertebra bone) interrogation. The Smart Tool Probe is a hand-held device directly used to interrogate the tissue site. It utilizes a system of optical fibers that carry the interrogating optical signal sent by the light source(s) and the reflected optical signal back to the optical receivers. The light source(s) and light receivers are located in the EOC Module. The data received from the EOC Module are processed and converted into an image which is displayed on the screen in real-time. The software installed on the machine allows the surgeon to adjust/enhance the image properties to suit the selected requirements. This mode of operation provides interactive image sharpening (to adjust image sharpness), threshold control (to adjust image contrast), segmentation (to delineate the density map in the image), image calculus (to pin-point the center of a particular region in the image) etc.

The system is contained in a hand held tool that can be used by a surgeon to identify the correct entry site and trajectory angle for cannulation (drilling a passage through) a spinal pedicle. The hand tool uses a light source to penetrate and interrogate the bony surface and the bone volume of the spine and collects and relays information to the surgeon regarding the bony topography beneath the instrument tip. This allows the surgeon to select the ideal starting point and trajectory for the placement of a passage through the pedicle.

The device can be manufactured as a reusable or disposable tool or instrument. Importantly, the tool is designed for surgeons who are familiar with other instruments or tools such as surgical awls or curettes. This should significantly reduce the usual learning curve associated with the use of new technology. In one embodiment, the device has a profiled scanning head enabling matching of the device to the actual shape of the interrogated bone. In another embodiment, the device is capable of real-time switching between various modes of operation of the interrogating optical wave, for example, DC mode, and modulation of amplitude, phase and frequency to optimize the device to the actual surgery operating conditions.

The system allows visualization imaging of bone (e.g. pedicle) in difficult situations where current techniques are deficient, including obesity, revision surgery, osteopenia/osteoporosis or small pedicles. The system provides a means to diagnose and monitor osteopenia/osteoporosis/osteopetrosis. The system also provides a means to diagnose, localize and stage bony tumors (metastatic or primary). The system also can be used as a means to diagnose and localize non-unions- or pseudarthroses and pseudarthrosis of the bone. The system can be used for evaluation of the surgical procedure during and after surgery, and for a long term monitoring of the integrity of the screw placement as well other accompanying effects such as bone cracking, etc.

### BRIEF DESCRIPTION OF THE DRAWINGS

FIG. 1A is a Smart Surgical Tool Block diagram. FIG. 1B is a schematic of incident, transmitted and scattered light during an interrogation of a vertebra by an optical beam. The disclosed device is depicted as a scanning position of an optical beam delivered by a fiber, for detection of the entrance to a pedicle.

FIGS. 2A and 2B are schematics of an optical pedicle probe with two different configurations of fibers.

FIG. 3 is a schematic of a smart surgical drill probe with embedded fiber optic sensors and accompanying optoelectronic circuitry placed in the grip of a drill.

FIGS. 4A-4F are schematics of lateral scanning (A,D), pedicle found (B,E), and access channel created (C,F), comparing the intrinsic mode (sensor and receivers within drill probe (A-C)) and extrinsic mode (sensor in drill, receivers outside of drill (D-F).

FIGS. 5A, 5B, 5C, and 5D are diagrams of a functional lay-out of a smart surgical drill, fiber optic probe Y configuration (5A), fiber optic probe T configuration (5B), fiber optic probe circular arrangement with center source (5C), and fiber optic probe circular arrangement with center detector (5D).

FIG. 6 is a perspective and cross-sectional view of a smart surgical drill probe with an accompanying optoelectronic detection system.

FIGS. 7A and 7B are block diagrams of a wireless measurement system for a Smart Pedicle Probe.

FIG. 8 is a block diagram of a fiber optic pedicle detection system (LFOPDS)

FIG. 9 is a perspective view of a Smart Tool, showing optical fibers.

### DETAILED DESCRIPTION OF THE INVENTION

### I. Definitions

As used herein, “smart” refers to an interactive device that transmits, receives and responds to information.

As used herein, “information” is a signal that provides information. The signal may be electrical, ultrasonic, laser (or light), radio, or other means of transmission of data.

As used herein, an “optical fiber” is any conduit through which light can be transmitted, either from a source, or as reflected, scattered, transmitted or diverted by or through a material, such as bone, cartilage or other tissue.

As used herein, an “optical source” is any optical source such as A laser, optical diode, active fiber, hybrid system emitting monochromatic or multi-wave length light, of different frequencies or wavelengths, including visible, infrared and ultraviolet range, continuously or modulated in amplitude (continuous, pulse modulation), phase and frequency

As used herein, an “optical receiver” is any optical energy receiving element/device such as A photodiode, phototransistor, optical integrating circuit, hybrid system capable of receiving monochromatic or multi-wavelength light signals, of different frequencies or wavelengths, including visible, infrared and ultraviolet range, continuously or modulated in amplitude (continuous, pulse modulation), phase and frequency

As used herein, a “computer” is any device capable of analyzing information, optionally storing and displaying the information.

As used herein a “surgical drill” probe is a device capable of penetrating bone, cartilage or other tissue by removal of a portion of the bone, cartilage or other tissue.

### II. Smart Surgical Tools and Systems

A probe for use with surgical tools has been developed to provide real time feedback while the tools are being used. Representative surgical tools include drills, probes, awl, needle, trochar, curette, or other similar instruments.

The probe includes the following features:

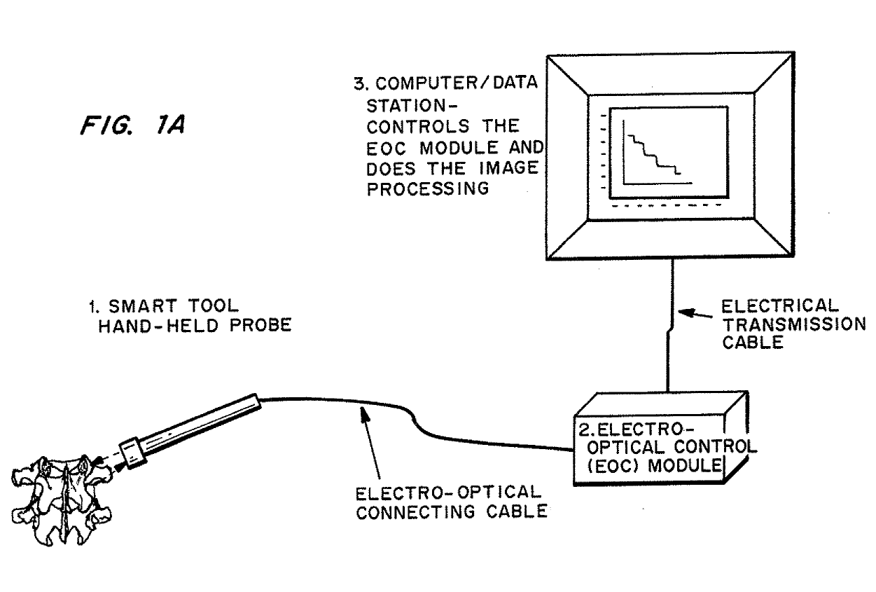

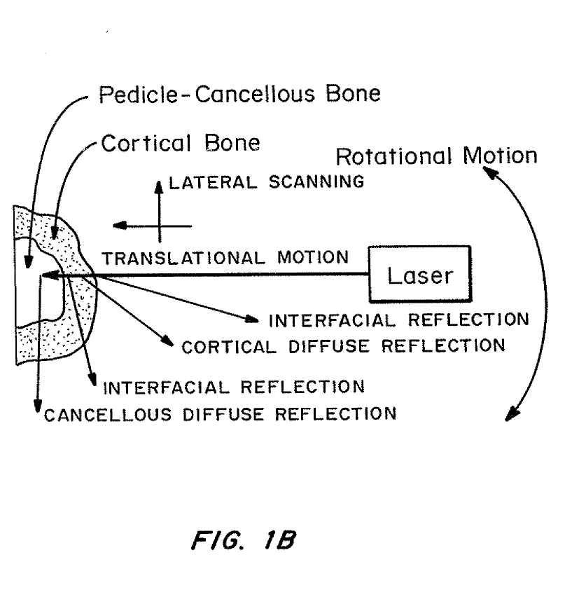

The Smart Tool is an interrogation system includes Smart Tool Probe and two processing modules, as depicted in FIG. 1A. Smart Tool Probe is hand held, wired or wireless, device that a surgeon utilizes for interrogating and identifying the entrance to a pedicle. The processing units, Electro-Optical Control (EOC) Module and Computer/Data Processing (CDS) Module provide a necessary control and display capabilities enabling real-time vertebra bone interrogation.

The Smart Tool Probe is a hand-held device directly used to interrogate a vertebra. It utilizes a system of optical fibers that carry the interrogating optical signal sent by the light source(s) and the reflected optical signal back to the optical receivers. The light source(s) and light receivers are located in the EOC Module. In another design all functional interrogating components that include optical fibers, optical sources, and optical receivers are located in the Smart Tool Probe.

The system is designed to provide a non-invasive imaging system enabling navigation of a tissue such as the pedicle region of the vertebra, having the features of:

A hand-held device for ease and interactive use.

An easily maneuverable tool that will enable the surgeon to navigate in the pedicle region of the vertebra.

A tool that generates a real-time image of the bone density distribution in the pedicle region of the vertebra, providing images of the cancellous and cortical regions in the pedicle.

A tool that provides a visual marker on the bone surface for drilling a hole for screw insertion with pin-point precision.

A wireless mode of operation enhancing convenience and easy to operate capabilities of the Smart Tool Probe,

A disposable and inexpensive Smart Probe Head to avoid cross-contamination issues.

A hand grip made of a material such as a polypropylene blend that can be steam sterilized or irradiated.

Electro-Optical Control (EOC) Module:

The EOC module controls many functions/processes within the Smart Tool Probe. It controls the switching between the source and the detectors, so that one obtains the pixel data from each point along the Probe interrogating directions. It also converts the optical signal to an electronic signal and transmits an electronic signal using wired or wireless mode which next is processed in the computer/data station.

The EOC includes many optical and electronic components such as optical light sources (lasers/LEDs), optical photo-detectors, electronic circuitry to drive the switching between the source and the detectors as well as a signal processing unit.

Optical sensors are included in the surgical device to provide a non-invasive, sensitive and reliable means measuring the location of and physical and biochemical properties of bones. Sensors are generally based either on measuring an intensity change in one or more light beams or on looking at phase changes in the light beams by causing them to interact or interfere with one another. Sensors are termed either intensity sensors or interferometric sensors. Techniques used in the case of intensity sensors include light scattering (both Rayleigh and Raman), spectral transmission changes (i.e., simple attenuation of transmitted light due to absorption), microbending or radiative losses, reflectance changes, and changes in the modal properties of the fiber. Interferometric sensors have been demonstrated based upon the magneto-optic, the laser-Doppler, or the Sagnac effects.

Optical sensors are available for measurement or control of various types of processes in virtually every field of applications. The basic advantage of optical sensors is that they offer a noninvasive, sensitive, rigid, and reliable mean of a measurement method compatible with electronic signal processing and data acquisition systems. The proven success of biomedical optical sensors results from their reliability and biocompatibility and the simplicity of the sensor-physician interface. Both invasive and noninvasive types have been developed and manufactured. For the most part, sensors are currently based on silica or plastic fibers that are coupled to sensitive sensors called optrodes, and utilize intensity modulation interrogation schemes.

The devices described herein utilize integrated fiber optic-based vertebra interrogating sensors operating under dual-mode conditions: intrinsic and extrinsic modes. The dual-mode operations increase the reliability of the measurements and provide detection adaptability and operational flexibility of the device to the complex and variable patient-specific conditions that a surgeon typically encounters during surgical procedures.

Computer/Data Processing Module:

The data received from the EOC Module are processed and converted into an image which is displayed on the screen in real-time. The software installed on the machine allows the surgeon to adjust/enhance the image properties to suit the selected requirements. This mode of operation provides interactive image sharpening (to adjust image sharpness), threshold control (to adjust image contrast), segmentation (to delineate the density map in the image), image calculus (to pin-point the center of a particular region in the image) etc.

Tool Design

Although described with specific reference to probes for placement of implants within pedicles, it will be understood that the technology is applicable to other types of surgical devices, as discussed above.

When an optical beam is incident on a vertebra, the light is transmitted, reflected and scattered from the subsequent structural components of the vertebra. This is depicted in FIG. 1B. There is incident, transmitted and scattered light during an interrogation of a vertebra by an optical beam. The scanning position of an optical beam delivered by an optical fiber is very convenient for detection of the entrance to a pedicle. Ideally, the optical detection and monitoring systems is able to receive all those reflected and scattered components of the optical interrogating wave, and convert them into the relevant information that subsequently is delivered to a surgeon.

The “Optical Smart Tool Technology” utilizes integrated fiber optic-based vertebra interrogating sensors operating under dual-mode conditions: intrinsic and extrinsic modes. The two-mode operations increase significantly the reliability of the measurements and provide a necessary detection adaptability and operational flexibility of the tool to the complex and variable patient-specific conditions that a surgeon usually encounters during surgical procedures.

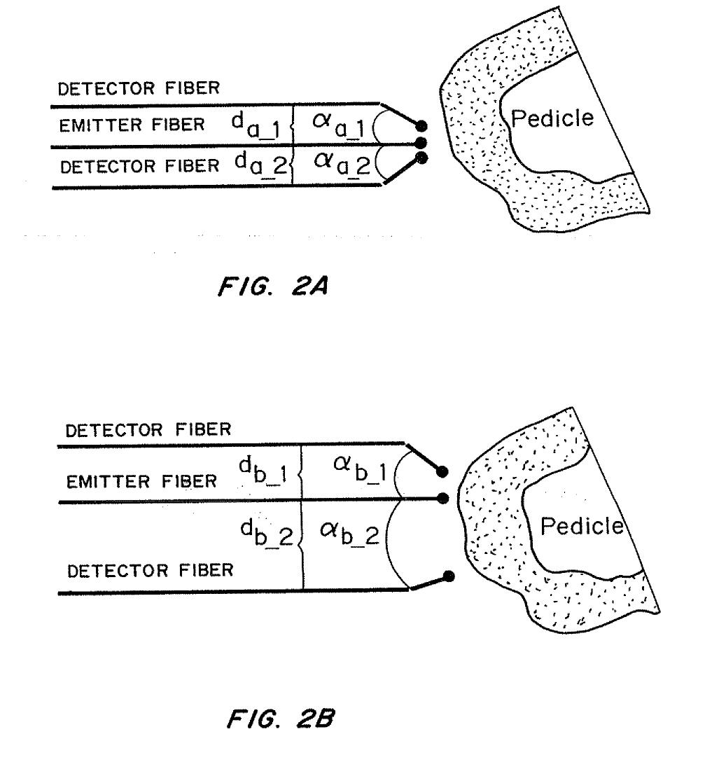

The important design parameter is the spatial sensitivity of the fiber optic probe to the location of a pedicle. This sensitivity is determined mainly by four design factors of the fiber optic probe. The first factor is related to the functional arrangement of fibers, i.e. the sensitivity is a function of whether or not the source fiber is in the center of the probe or is/are placed on the perimeter. The second factor depends on the angular position of optical fiber receivers with respect to the central fiber, i.e. angles α₁ and δ₆₂ in FIGS. 2A and 2B. The third factor is related to the actual number of the fibers employed in the probe. Three or five fibers are currently preferred. The number of fibers depends on an actual specific need and can range from a single fiber to several hundreds. The fourth factor applies to an overall system performance related to the accuracy by which one can measure the changes caused by the presence of a pedicle. This relates to the opto-electronic measurement technique and the overall measurement accuracy, i.e. how accurately one can measure the reflected light and determine the spatial position. These in turn depend on the signal to noise ratio of the output signal. This ratio is influenced by the sensitivity of the output signal to the ambient conditions such as temperature, humidity, vibrations, and electronic circuitry design. For example, one can use the constant power optical source (simple and inexpensive) or one can modulate the interrogating optical source, to improve signal to noise ratio and increase the dynamic range of the detection system.

Important operational parameters of a fiber optic probe are determined by the spatial arrangement of the fibers. In a first arrangement, the central fiber acts as a source fiber and emits an interrogating optical wave into a bone, and the peripheral fibers act as the receivers of the reflected wave. In a second arrangement, the central fiber acts as the receiver and the interrogating wave is launched from the peripheral fibers. It is likely that these will provide complementary information. In such a case, with a slight modification of electronic circuitry (electronic switch), one can utilize two methods simultaneously. Also, the angle at which the optical wave is launched and received is critical for the design of the probe. The angular dependence of the incident and reflected wave characteristics over the full 180° angle range change can be utilized. The number of the fibers used for launching and reception of optical wave will impact technical features of the probe including sensitivity, dynamic range, spatial resolution and accuracy in determination of the entrance to the pedicle. A CCD strip may be used instead a discrete number of fibers.

In another arrangement the light is emitted from a fiber at a given location, and the received light is collected from the fibers located at predetermined distances from the emitting fiber. By changing the placement distance of the receiving fibers, the received light comes from different depths of the bone therefore receiving the images of the bone at different depths and next, by integrating those individual slicing responses to create three dimensional images of the bone.

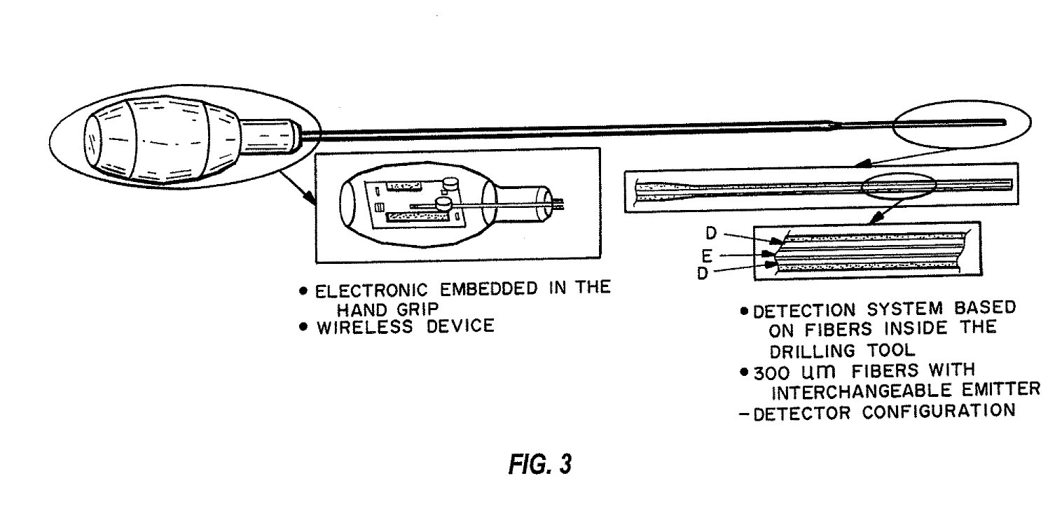

An embodiment of a smart drill 10 is depicted in FIG. 3 in which a fiber optic sensor system is integrated with the surgical rod 30 and an accompanying optoelectronic circuitry 36 is located in the grip 32 of the drill. Optical fibers are embedded directly in the surgical tool 10. At least three optical fibers 12, 14, 16 are placed in drilled or molded internal conduits 18, 20, 22 made inside the tool, as depicted in FIG. 3. One fiber 12, preferably placed in the center 18 of the tool 10, delivers an optical interrogating signal to the bone, and two other fibers 14, 16, located at the perimeter of the drill, are utilized as the optical receivers of the optical wave scattered from the bone. The surgical tool can be designed exactly as a typical surgical drill except that the stainless steel rod is replaced with a rod made of soft metal (e.g. brass) in order to facilitate easy machining necessary for integration of the fiber optic pedicle detection system. The hole drilled along the center of the rod supports a fiber operating as a center emitter or receiver; on the side of the rod, two or four fibers can be attached for either sending or receiving optical wave. An important design consideration is that the sensor arrangement can not interfere with a typical tool handing procedure performed by a surgeon. Another critical design factor is to minimize the weight of the fiber system and accompanying electronics.

If the probe response is too strongly influenced by ambient conditions, this could affect repeatability and signal-to-noise ratio. Three alternative approaches could be used in such a situation: (i) a different design of the fiber attachment to the central rod of the probe and other shapes and different techniques for bonding of the lever; (ii) a different design of the tip of a rod, which should improve the sensitivity and the signal-to-noise ratio because a dedicated tip can protect the fibers; (iii) the use of a higher optical power of optical sources or an AC modulated optical source versus a DC optical wave which will enable utilizing a phase-lock-loop (PLL) approach. Though PPL systems are more complicated than a simple oscillator design, it should be more stable under demanding ambient conditions.

In the extrinsic mode, the intrinsic probe is aided by an additional movable fiber optic sensor head concentrically placed at the end of the drill. The head includes optical fibers placed along the perimeter of the sensor head and used as the receivers of the optical wave generated by the light emitting fiber placed in the center of the drill.

a. Intrinsic Mode

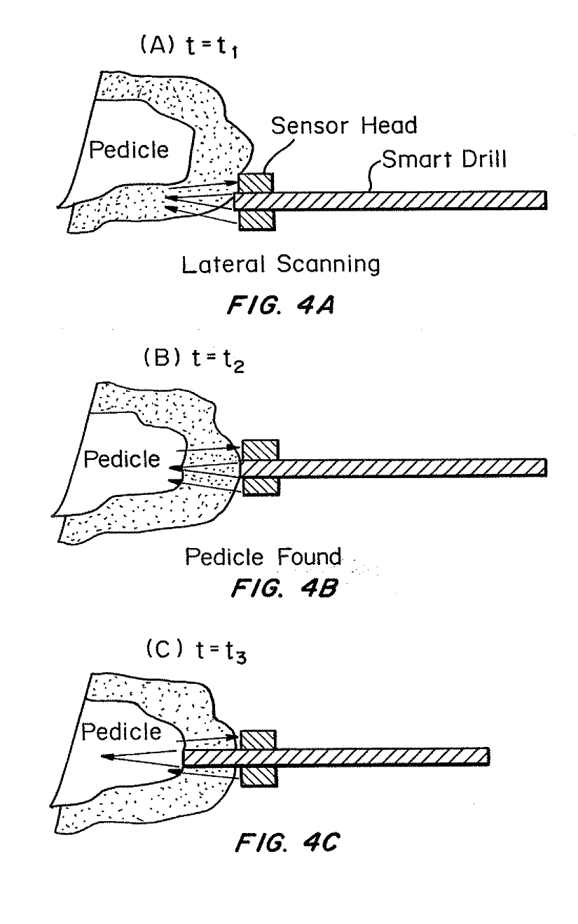

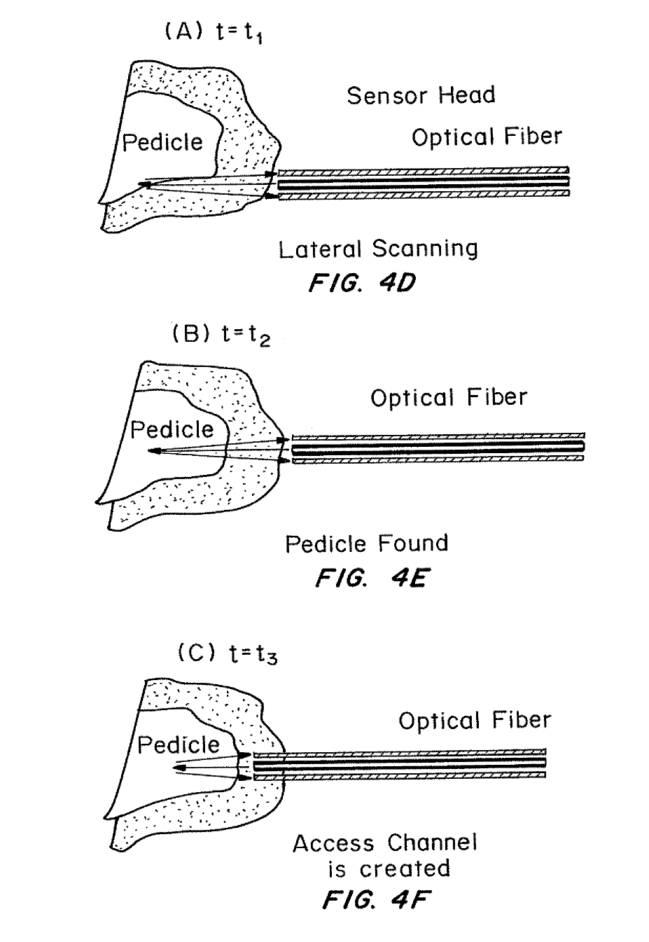

In the intrinsic mode, the optical fibers are embedded directly in the surgical tool. At least three (3) optical fibers are located in the drilled internal conduits made inside the tool (FIGS. 4A-E, 3). One fiber (FIGS. 3, 12), placed in the center of the tool (FIGS. 3, 10) typically delivers optical interrogating signal to the bone, and two other fibers (FIGS. 3, 14 and 16), typically located at the perimeter of the drill (FIGS. 3, 30), generally function as optical receivers of the optical wave scattered from the bone.

After lateral scanning of the surface of vertebra (FIG. 4A) and location of the entrance to a pedicle (FIG. 4B), the drill moved by a surgeon provides information to a surgeon on the bone properties as well the trajectory of the movement of the device taking place in inside the vertebra (FIG. 4C).

b. Extrinsic Mode

In the extrinsic mode (FIGS. 3D-F), the intrinsic probe is aided by an additional movable fiber optic sensor head. Typically, the movable fiber optic sensor head is concentrically located at the end of the drill. The movable fiber optic sensor head includes optical fibers placed along the perimeter of the sensor head. These optical fibers serve as the receivers of the optical wave generated by the light emitting fiber placed in the center of the drill (FIG. 4D).

After finding the entrance to a pedicle (FIG. 4E), the movable fiber optic sensor head will remain outside the vertebra, receiving the optical signals coming from the inside of the vertebra (FIG. 4F). The information will complement the data received, at the same time, from the intrinsic mode of operation of the drill.

In a preferred embodiment, the fiber arrangement includes a central fiber encircled by the peripheral fibers distributed along a perimeter.

TABLE 2
Fiber Optic Design

| | Number | Fibers | | Configuration |
|---|---|---|---|---|
| N° | of Fibers | Arrangement | Side View | Advantages |
| 1 | 3 Fibers | 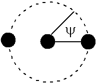 | 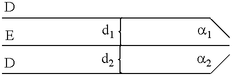 | •Easy to Implement. |
| 2 | 3 Fibers | 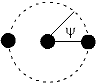 | 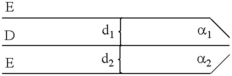 | •Rapid Results. |
| 3 | 5 Fibers | 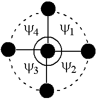 | 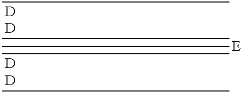 | •High Resolution. |
| 4 | 5 Fibers | 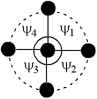 | 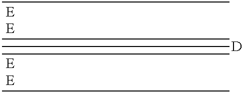 | |

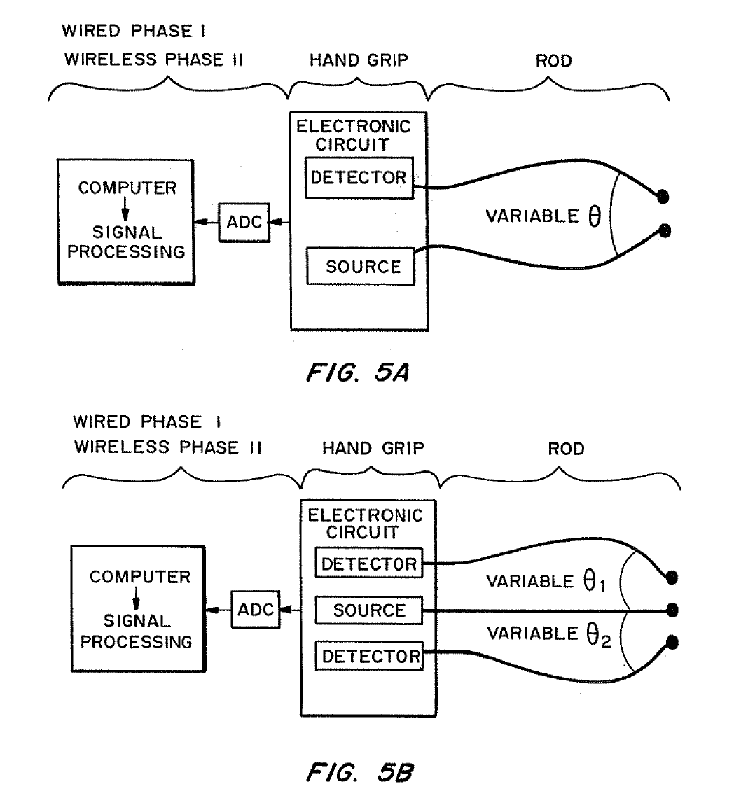

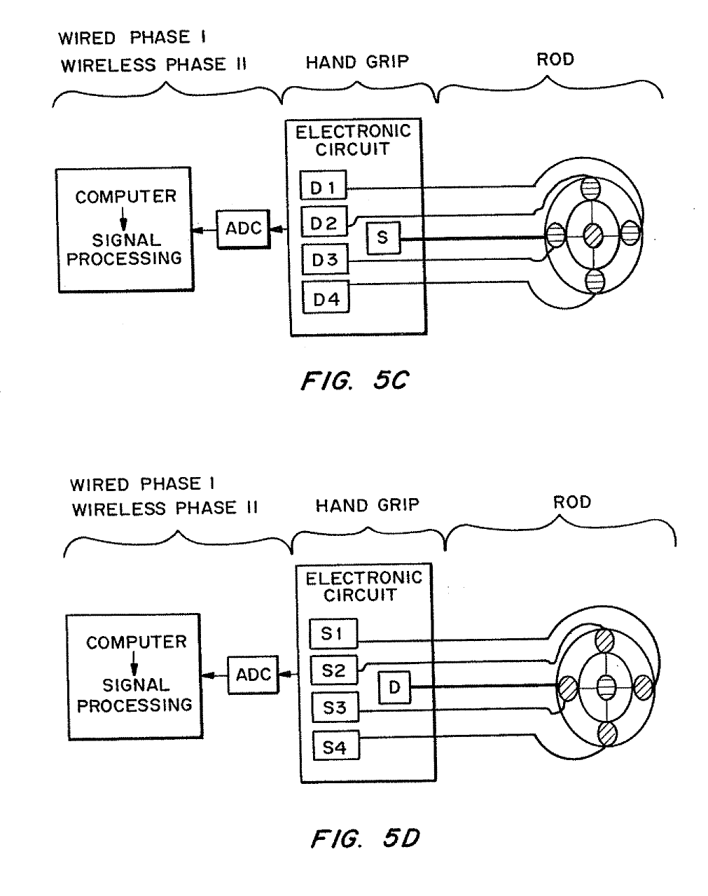

FIGS. 5A-5D depict a variety of different source and detector fiber optic arrangements that can be used. FIGS. 5A and 5B reflect three fiber optic configurations. In FIG. 5A, the source and detector are in a Y configuration extending from the handgrip. In FIG. 5B, the source is in the center and there are two (the same or different) peripheral detectors, referred to as the ψ configuration. FIGS. 5C and 5D reflect five fiber optic configurations. In FIG. 5C, there are five fiber optics, four peripheral detectors and a center source. In FIG. 5D, there are four peripheral sources and a central detector.

System Requirements

The surgical tools are packaged with disposables in a sterilized package for ready use in the operating room or out patient clinic. These are integrated into a system including a power source, amplifiers and digital displays (a computer monitor) and hardware and software package for visualization and analysis of the data received from the sensor(s), and in the case of powered tools, a source of power/force to operate the tool.

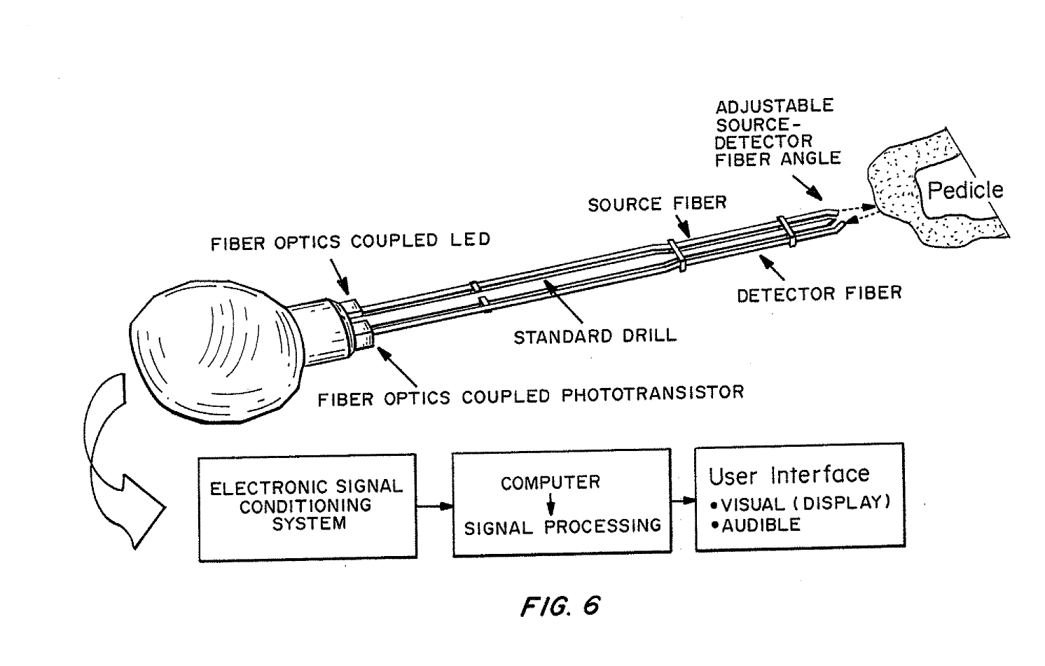

FIG. 6 is a schematic of the smart drill 10 with drill 30 containing source fiber 12, detector fiber 14, and an adjustable source detector fiber angle 24. This is connected to fiber optics coupled light emitting diode (“LED”) 40 and/or phototransistor 42 in the drill handle 32. The drill is integrated with system components via an electronic conditioning system 46, which sends signal to a computer 48 or other data or signal processing device 50 which provides an user interface 52, typically a visual display 54 and/or audible display 56.

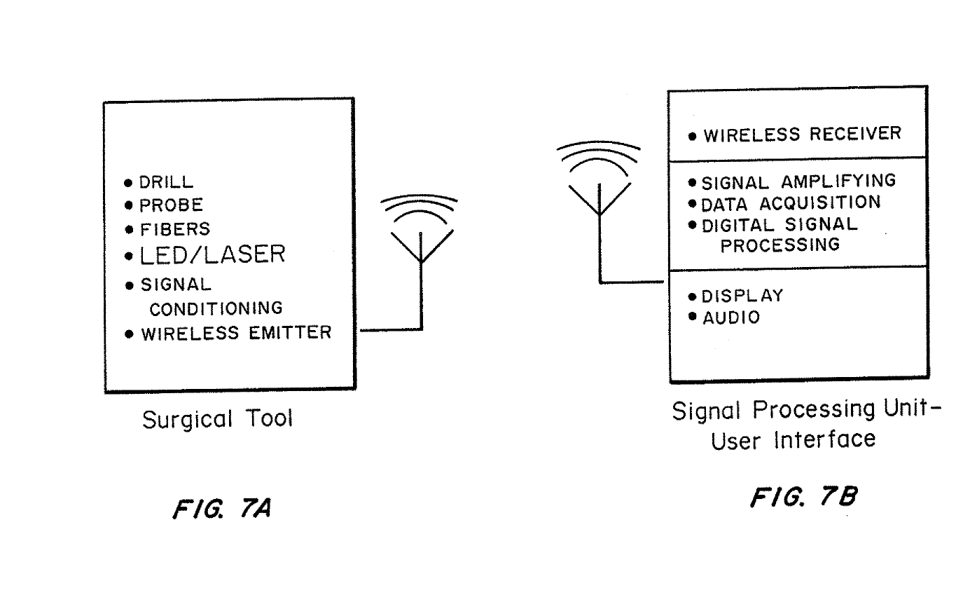

In a preferred embodiment, the system is wireless, as depicted in FIGS. 7A and 7B. The tool unit including drill, probe, fibers, LED/LASER, signal condition, is connected with a wireless emitter; the signal processing unit-user interface includes a wireless receiver connected with components for signal amplifying, data acquisition, digital signal, and processing, which is then displayed visually and/or in audio form.

A commercial wireless communication system such as MOTOROLA® BE-304 provides the necessary operational functionality that implements all measurement functions. A simple audio-visual signaling indicating the presence of the entrance to the pedicle may also be utilized.

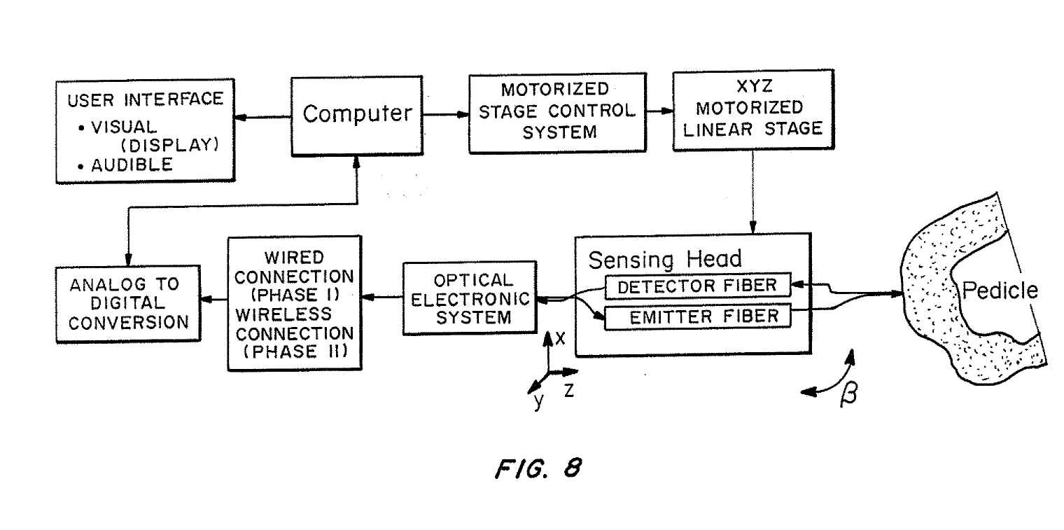

FIG. 8 is a system block diagram of the entire system. The system may be modular or integrated. In addition to the components discussed above with respect to the surgical tool, wireless or wired connections, and computer and user interfaces, one could include a motorized stage control system and an XYZ motorized linear stage to facilitate handling and control of the tool.

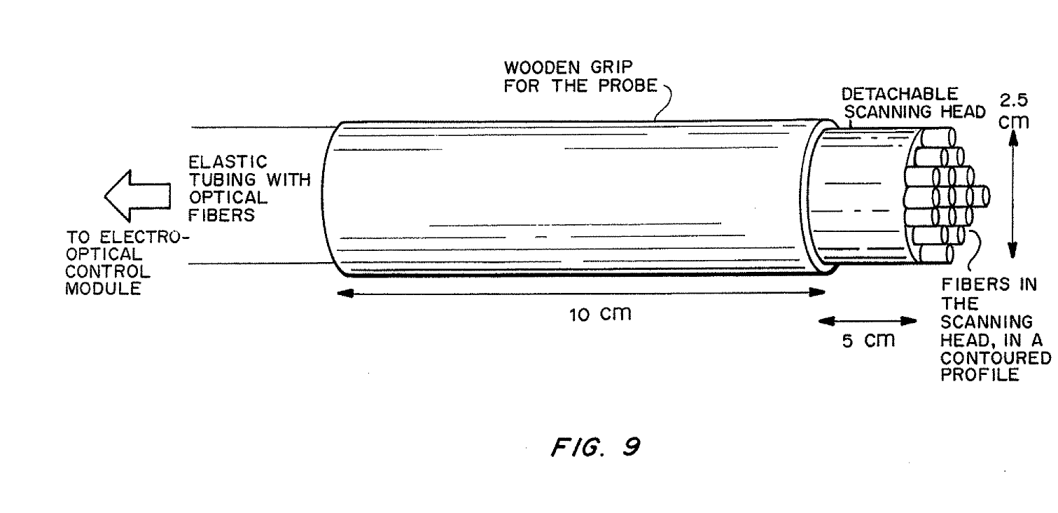

A preferred embodiment is depicted in FIG. 9. The Smart Tool Probe consists of the Base Probe (BP) and Disposable Scanning Probe Head (DSPH). The BP consists of a tubular casing which holds a bundle of transmitting and receiving optical fibers that terminate into an interface with the fibers located in the DSPH. The optical wave transmitted by the DSPH interacts directly with the bone. There are various types of the DSPH designs depending on a specific application i.e. the region of interest (lumbar versus cervical vertebra), type of patient (children versus adults), etc. The fibers inside the DSPH are arranged in different configurations (for example, all the fibers in one plane, or contoured and profiled to the bone surface geometry) in order to suit the different interrogating needs of the Smart Tool. Finally, an ergonomic grip in combination with a flexible tube carrying optical fibers allows a surgeon to maneuver the Smart Tool with ease and convenience, and high accuracy and reproducibility of interrogation.

In a preferred embodiment, the functional interrogating components including optical fibers, optical sources, optical receivers and EOC module are located in the Probe. The functional interrogating components including optical fibers, optical sources, optical receivers and EOC module located in the probe operate over a broad range of optical spectrum such as visible, infrared, UV and other frequencies of electromagnetic spectrum. The tool can contain multiple optical fibers, at least one of which is for transmission of light and at least one of which is for receiving reflected light. In one embodiment, at least one fiber is in the center and serves as a source of light, and at least one fiber placed elsewhere relative to the light source, and serves as a receiver. In another, at least one fiber is in the center and serves as a receiver of light, and at least one fiber is on the perimeter of the device relative to the source, and serves as a source of light. The angle of the fiber optic source and the fiber optic receivers can be adjusted and/or optimized for the material to be detected using optical lens, prism or optical deflecting system. Alternatively, the interrogating angle of the fiber optic sources and the acceptance angle of the fiber optic receivers can be adjusted and/or optimized for the material to be detected by creating a length-varying distribution of the fibers in the probe thus producing a variable fiber profile of the probe.

The system includes optical emitters, optical receivers, electro-optical multichannel driving units, signal conditioning units, signal processing units, and multimodal representation of the information on the bone in form of visual signal like screen or audio signal.

Although described herein primarily with respect to the use of light for measurement and interrogation, other modalities could also be incorporated as used to provide additional or alternative information. For example, the device could utilize ultrasound as a means for measurement.

### III. Methods of Using Device

The system provides a means of utilizing light energy for determination of structural features of a bone or tissue, utilizing light of different wavelengths, energy, frequency and polarization for determination of structural features of the tissue or bone. This allows light of variable energy to be used to determine structural features of the tissue or bone at different depths from the bone surface. This also provides a means of making a two or three dimensional image of the tissue or bone.

This is particularly useful in imaging of bony areas such as the pedicle, where the light energy is used to determine the entrance to a pedicle and the trajectory angle of the pedicle's course to the vertebral body. The system also provides a means for utilizing light energy for determination of mechanical properties of the bone such as mechanical strength, bone integrity, and bone-mineral density. This in turn makes the system useful for determination of bone diseases such as cancer, chronic infection, cysts, avascular necrosis, inflammatory tumors such an osteoid osteoma, and for other purposes, such as identifying fibrocartilage defects indicating non union within a surgical fusion mass or pseudoarthrosis and interrogation of the tissue surrounding the tissue or bone. The system also provides a means for identification of the type of the tissue surrounding the bone, blood vessels, or nerves.

The system allows visualization imaging of bone (e.g. pedicle) in difficult situations where current techniques are deficient, including obesity, revision surgery, osteopenia/osteoporosis or small pedicles. The system provides a means to diagnose and monitor osteopenia/osteoporosis/osteopetrosis. The system also provides a means to diagnose, localize and stage bony tumors (metastatic or primary). The system also can be used as a means to diagnose and localize non-unions or pseudarthroses and pseudarthrosis of the bone. The system can be used for evaluation of the surgical procedure during and after surgery, and for a long term monitoring of the integrity of the screw placement as well other accompanying effects like bone cracking, etc.

To use the device for localization of the spinal pedicle, the surgeon would move the tools, instrument or smart drill (“smart tool”) along the surface of the bone. The light emitted from the smart tool will be transmitted to the bone, and the reflected light will be detected and transduced to a remote processing and monitoring device, providing the surgeon feedback as to when the smart tool tip is located directly above the center point of the spinal pedicle. The information can be relayed to the surgeon in a visual format (i.e. picture of the underlying bony structure), audible format (i.e. tone change as the smart tool tip passes over the center of the pedicle) or tactile (i.e. vibration or similar tactile sensation transmitted to the surgeon as the smart tool tip passes over the central region of the pedicle).

As soon as the center of the pedicle is identified, the smart tool will be made to penetrate the bone in that location. As the bone is penetrated, the smart tool will continue to give the surgeon feedback on whether the trajectory of the smart tool is in line with the central axis of the pedicle using visual, audible or tactile feedback. The surgeon will continue to penetrate the bone using the smart tool until a safe passage through the pedicle is achieved. At this point, the smart tool can be withdrawn and additional preparation for the pedicle implant (such as tapping the hole) can be undertaken. Alternatively, the smart tool can have a cannulation passage through its central section so that upon identification of the central axis of the pedicle, a guide wire can be placed through the smart tool, into the bone of the pedicle to act as a marker for the correct site and trajectory of the pedicle. The guide wire can then be over drilled and/or tapped using conventional means, known to the field of spinal surgery to prepare the pedicle for implant insertion. In an alternative embodiment, the smart tool can have a means to make a visible mark on the cortex of the bone at the ideal entry site into the pedicle. This mark can them be used to localize the site to open the cortex and enter the pedicle using conventional means, such as a high speed drill to breech to the cortex and enter the pedicle. The pedicle could then be probed along its length using a blunt pedicle probe device known to the field of spinal surgery and prepared for implant insertion.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A Smart Tool interrogation system comprising
a hand-held Probe,
an Electro-Optical Control (EOC) Module comprising a system of optical fibers that carry an interrogating optical signal sent by at least one light source to the interrogated object and receiving fibers that carry the received optical signal back to the receivers, and
a CDS Module providing control and display capabilities,
the system enabling real-time vertebra bone interrogation.

2. The system of claim 1 wherein the functional interrogating components including optical fibers, optical sources, optical receivers and EOC module are located in the Probe.

3. The system of claim 1 wherein the functional interrogating components including optical fibers, optical sources, optical receivers and EOC module operate over a broad range of optical spectrum selected from the group consisting of visible, infrared, UV and other frequencies of electromagnetic spectrum.

4. The system of claim 1 wherein the operating parameters of optical sources and interrogating optical wave including intensity, phase, and polarization are controlled and adjusted.

5. The tool of claim 1 comprising multiple optical fibers, at least one of which is for transmission of light and at least one of which is for receiving reflected light.

6. The tool of claim 1 wherein at least one fiber is in the center and serves as a source of light, and at least one fiber placed elsewhere relative to the light source, and serves as a receiver.

7. The tool of claim 1 wherein at least one fiber is in the center and serves as a receiver of light, and at least one fiber is on the perimeter of the device relative to the source, and serves as a source of light.

8. The tool of claim 1 wherein the angle of the fiber optic source and the fiber optic receivers can be adjusted and/or optimized for the material to be detected using optical lens, prism or optical deflecting system.

9. The tool of claim 1 wherein the interrogating angle of the fiber optic sources and the acceptance angle of the fiber optic receivers can be adjusted and/or optimized for the material to be detected by creating a length-varying distribution of the fibers in the probe thus producing a variable fiber profile of the probe.

10. The tool of claim 1 wherein the device is wireless.

11. The tool of claim 1 wherein the modules are both in the probe.

12. The tool of claim 1 comprising means for wireless transmission of data between the EOC and CDS.

13. The tool of claim 1 selected from the group consisting of drills, probes, awls, needles, trochars, and curettes.

14. The tool of claim 13 wherein the tool is a drill comprising the light source and the detector fiber remains outside of the hole being drilled to receive the input of light reflected from the fiber light source.

15. The tool of claim 1 comprising one or more disposable components, in a kit comprising tool and the one or more disposable kits.

16. A disposable sterile, separately packaged, covering or cutting tip for the tool defined by claim 1.

17. A system for use with the tool of claim 1 comprising means for data analysis and display, audio reception and transmission means, motorized linear stage or controls thereof.

18. A method utilizing light energy for determination of structural features of a bone or tissue comprising using the tool of claim 1, utilizing light of different wavelengths, energy, frequency and polarization for determination of structural features of the tissue or bone.

19. The method of claim 18 utilizing light of variable energy for determination of structural features of the tissue or bone at different depths from the bone surface.

20. The method of claim 18 comprising utilizing light energy for formation of a two-dimensional image of the tissue or bone.

21. The method of claim 18 comprising utilizing light energy for formation of a three dimensional image of the tissue or bone.

22. The method of claim 18 utilizing light energy for determination of the entrance to a pedicle and the trajectory angle of the pedicle's course to the vertebral body.

23. The method of claim 18 utilizing light energy for monitoring the movement of the surgical tool through the vertebral body

24. The method of claim 18 utilizing light energy for determination of mechanical properties of the bone such as mechanical strength, bone integrity, and bone-mineral density.

25. The method of claim 18 utilizing light energy for determination of bone diseases such as cancer, chronic infection, cysts, avascular necrosis, inflammatory tumors such an osteoid osteoma.

26. The method of claim 18 using light energy to identify fibrocartilage defects indicating non union within a surgical fusion mass or pseudoarthrosis.

27. The method of claim 18 in which the light used for interrogation of the tissue surrounding the tissue or bone.

28. The method of claim 18 in which the light is used for identification of the type of the tissue surrounding the bone, blood vessels, or nerves.

29. The method of claim 18 wherein the system comprises optical emitters, optical receivers, electro-optical multichannel driving units, signal conditioning units, signal processing units, and multimodal representation of the information on the bone in form of visual signal like screen or audio signal.
